(19) United States
(12) Patent Application Publication
(10) Pub. No.: US 2012/0269797 A1
(43) Pub. Date: Oct. 25, 2012

(54) METHOD FOR INHIBITING THE MATURATION OF DENDRITIC CELLS

(76) Inventors: Ulrich Schubert, Jena (DE); Elisabeth Zinser, Marloffstein (DE); Alexander Steinkasserer, Marloffstein (DE)

(21) Appl. No.: 13/384,024

(22) PCT Filed: Jul. 15, 2010
(86) PCT No.: PCT/EP2010/060265

§ 371 Date: Jul. 2, 2012

(30) Foreign Application Priority Data
Jul. 15, 2009 (DE) 10 2009 027 754.4

(51) Int. Cl. A61K 38/55; C07H 15/24; A61K 31/704; C07D 207/28; A61K 31/4015; C07K 5/083; C07F 5/04; A61K 31/436; A61K 31/5377; A61K 39/395; A61P 37/08; A61P 11/06; A61P 37/06; A61P 37/00; A61P 21/04; A61P 25/00; A61P 1/00; A61P 19/04; A61P 17/00; A61P 29/00; A61P 3/10; A61P 1/04; A61P 17/06; C07K 5/103

(52) U.S. Cl. 424/130.1; 530/330; 514/20.1; 536/17.2; 514/34; 548/534; 514/423; 530/331; 544/229; 514/291; 514/233.5

(57) ABSTRACT

Proteasome inhibitors (PI) are used for inhibiting the maturation of dendritic cells (DZ) and thus in the treatment or the prophylaxis of allergies, asthma, tissue or transplant rejection or autoimmune diseases. The concentration of the proteasome inhibitors lies preferably in the range of 10 nM to 10 μM, based on the peripheral blood or the cytoplasm.

The invention relates to the use of proteasome inhibitors (PIs) for inhibiting the maturation of dendritic cells (DCs) and thus for treating or preventing allergies, asthma, tissue rejections or transplant rejections, or autoimmune diseases.

### BACKGROUND OF THE INVENTION

The human and animal immune system is able to react to a large number of outside antigens. Lymphocytes play a central role in this, because they can recognize antigens and effectively stimulate the adaptive immune system. Lymphocytes can be divided into two general classes: B lymphocytes, which produce antibodies, and T lymphocytes, which are furthermore subdivided into CD4⁺ helper T cells and CD8⁺ cytotoxic cells. Furthermore, the so-called regulatory T cells, which can inhibit the function of/regulate the two other T cell types, are also included among them. All of them are able to recognize antigens that are presented on the so-called antigen-presenting cells (APCs), together with MHC molecules, by way of the T cell receptor (TCR).

The APCs can be subdivided into “simple APCs” that only present antigens, and “professional APCs” that possess stimulatory functions, on the basis of the expression of specific molecules, in addition to the antigen presentation. The best APCs known at this time are the “dendritic cells” (DCs). They are the only APCs that are able to stimulate naïve T cells and are therefore also referred to as a “natural adjuvant.”

Immature DCs are specialized in absorbing antigens, processing them, and installing them into the MHC complexes. It is significant in this connection that immature DCs are involved in maintaining or inducing tolerance mechanisms.

Stimuli such as TLR ligands, TNF, cytokines, CD40L, etc., are able to stimulate DCs to mature, leading to a massive synthesis of new MHC molecules and to the migration of the DCs out of the periphery into the draining lymph nodes. Furthermore, during DC maturation and DC migration in the lymph nodes, the expression of co-stimulating molecules, such as, for example, CD80, CD86, or CD40, as well as of adhesion molecules, such as, for example, LFA3, is also upregulated. After the matured DCs have migrated into the T-cell-rich areas of the lymph nodes, they present the MHC peptide complexes to the specific T cells and stimulate them to perform stimulation. MHC-I complexes presented in mature DCs stimulate cytotoxic T cells as well as Th17 cells, while T helper cells are stimulated by way of MCH-II complexes. In the presence of mature DCs and, among other things, IL-12, T helper cells differentiate to become Th1 specific T helper cells, which produce IFN-gamma, among other things. These subsequently support the differentiation of cytotoxic T cells. In contrast, IL-4 leads to the differentiation of Th2 cells, which activate eosinophils and B cells.

The phenotype of mature DCs can be checked by means of FACS analyses. In this connection, typical molecules (for example CD25, CD80, CD83, CD86, MHC-I, MHC-II, CCR7), which are upregulated during CD maturation, are detected.

### Task of the Invention

The invention was based on the task of making available new possibilities for treating/preventing allergies, asthma, tissue rejections or transplant rejections, or autoimmune diseases.

### Accomplishing the Task

The task was accomplished in accordance with the characteristics of the claims.

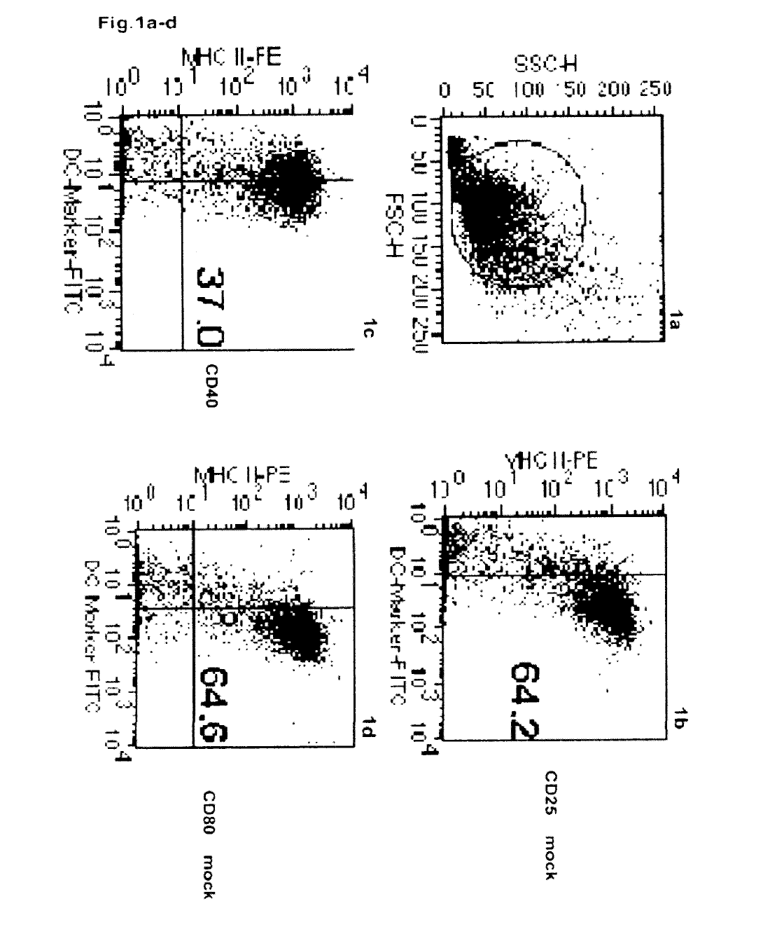

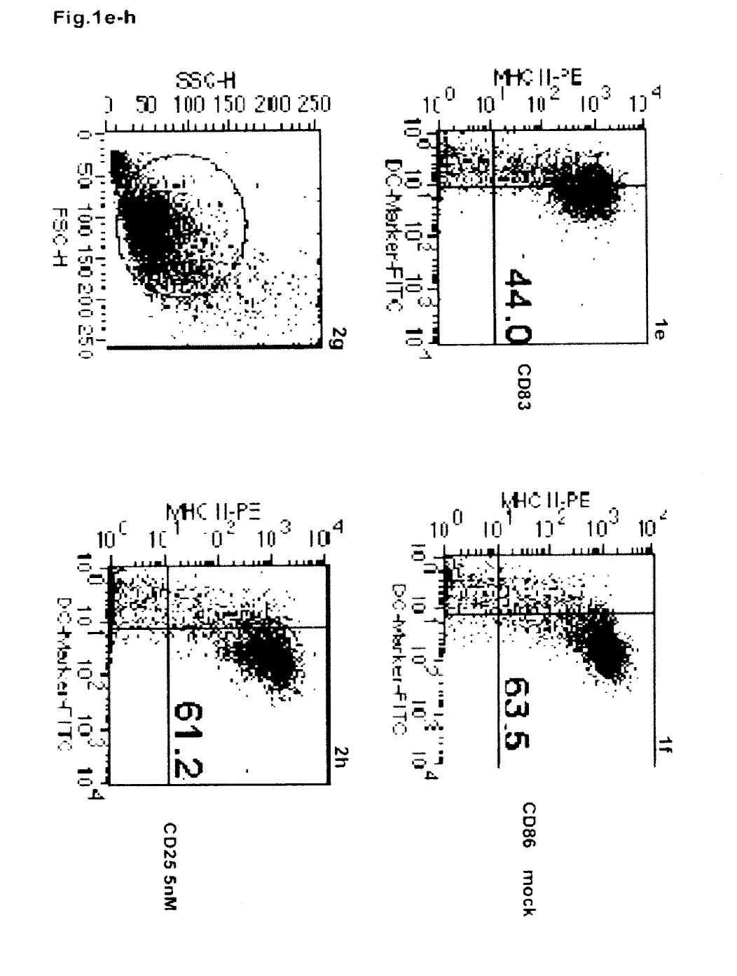

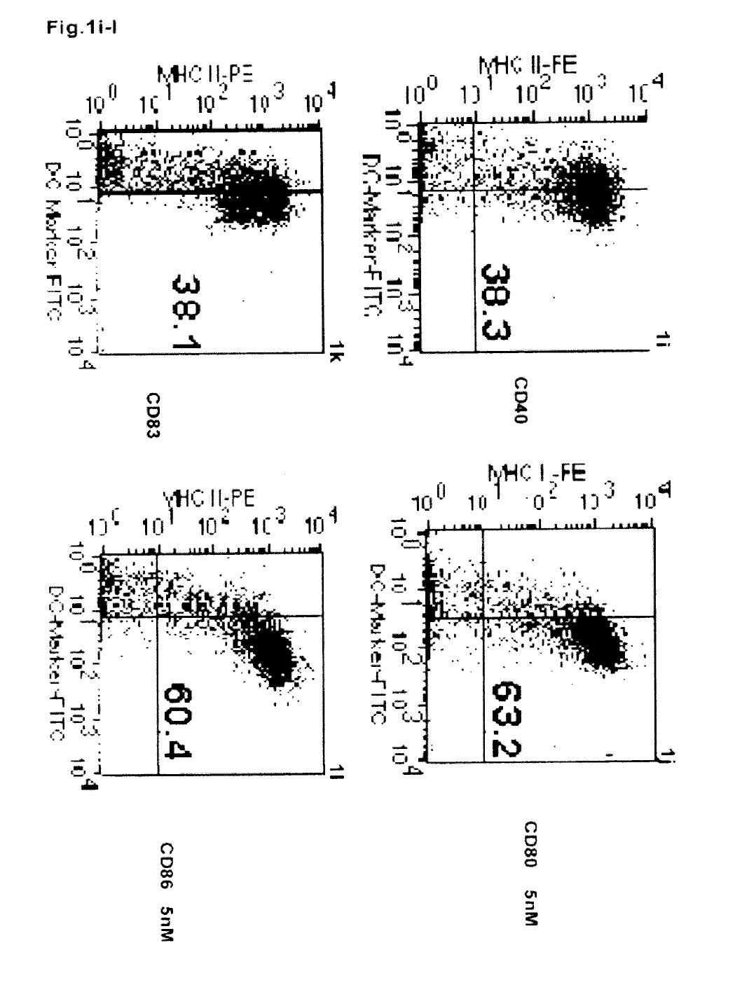

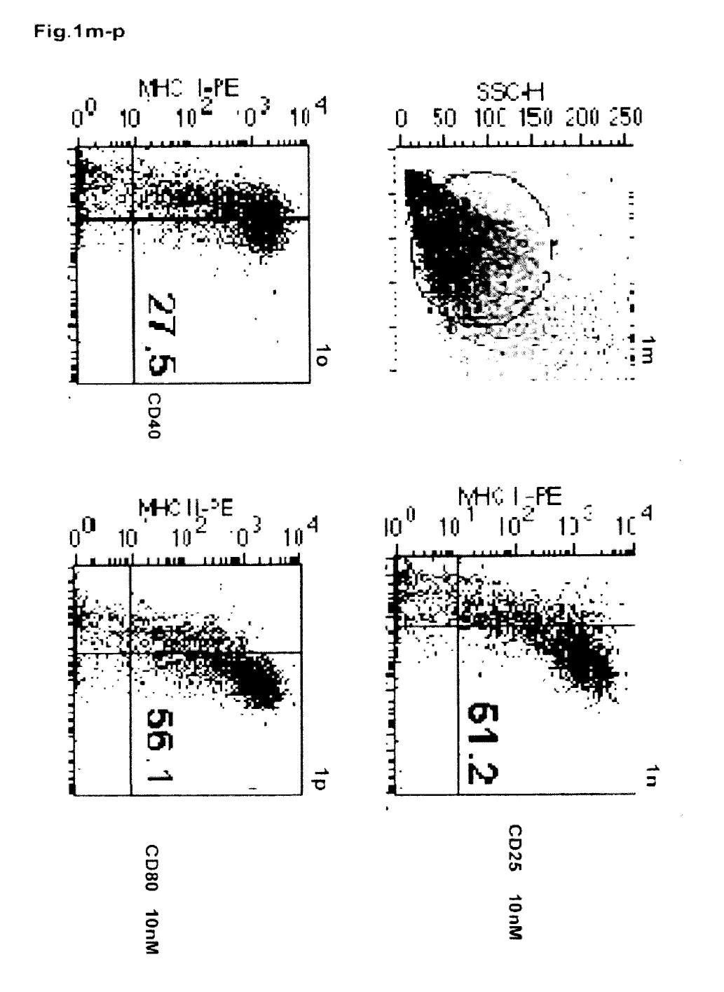

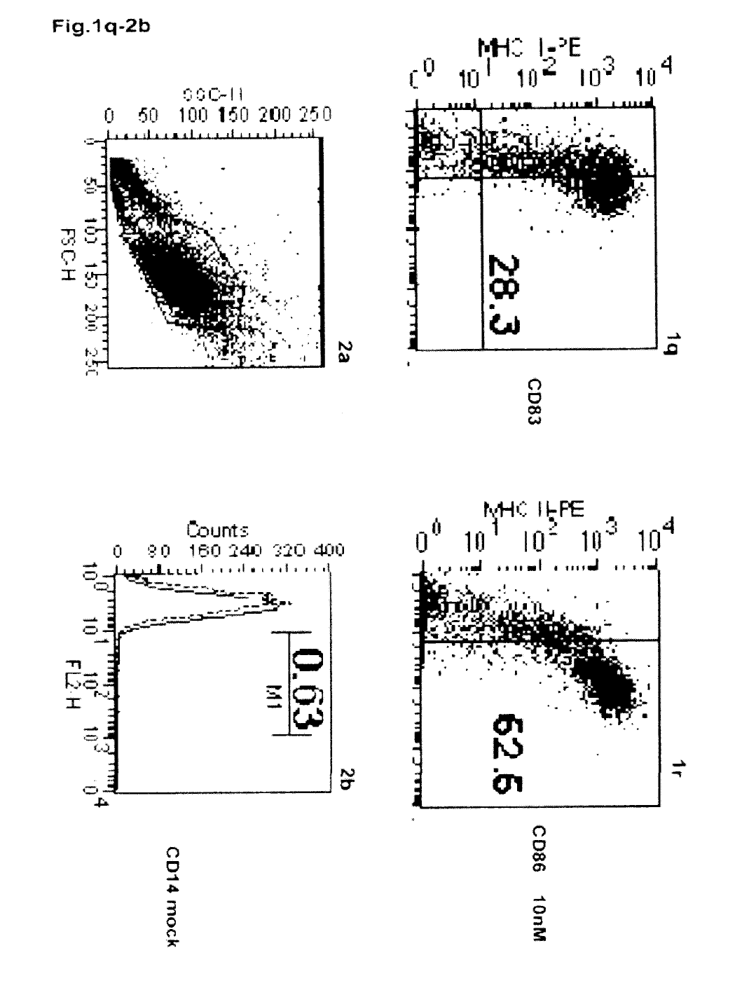

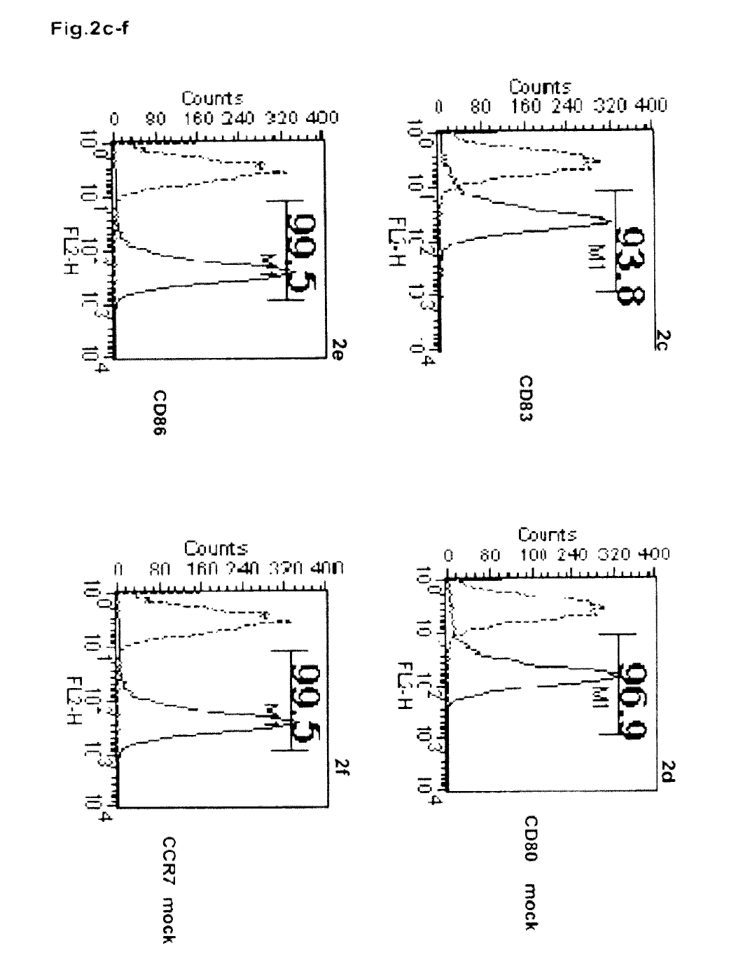

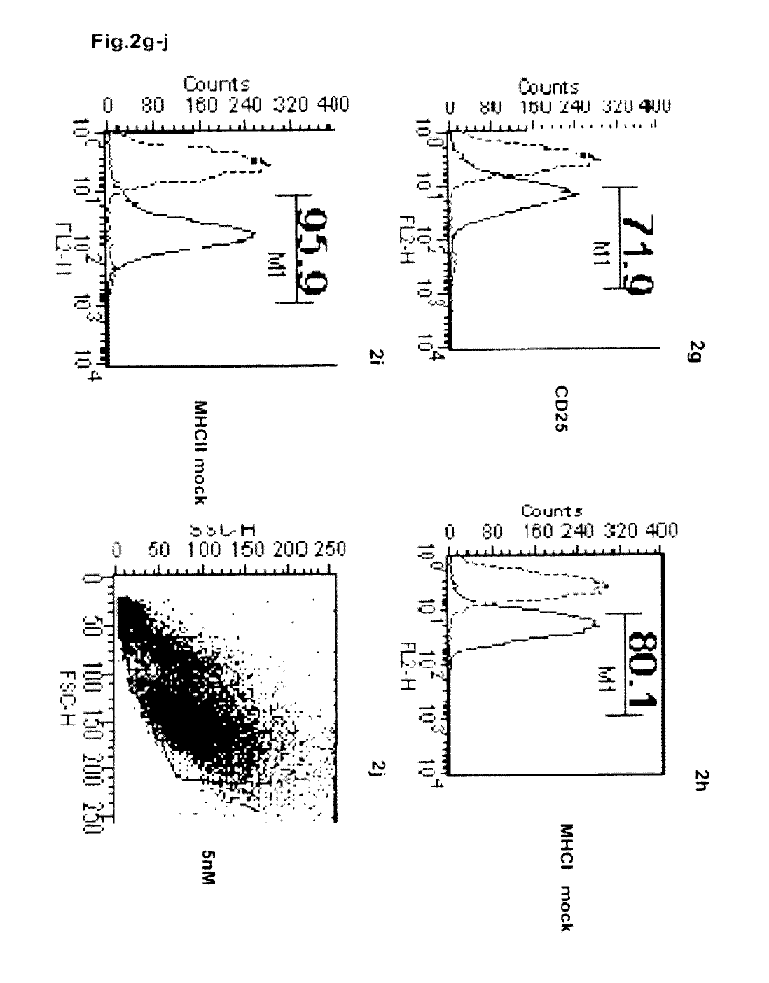

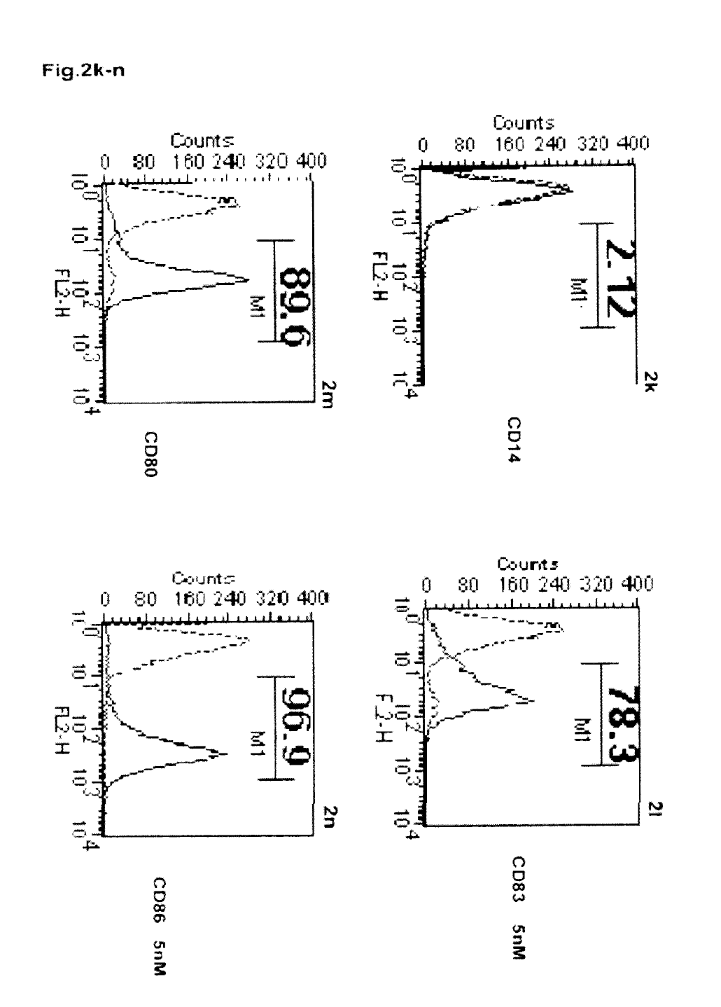

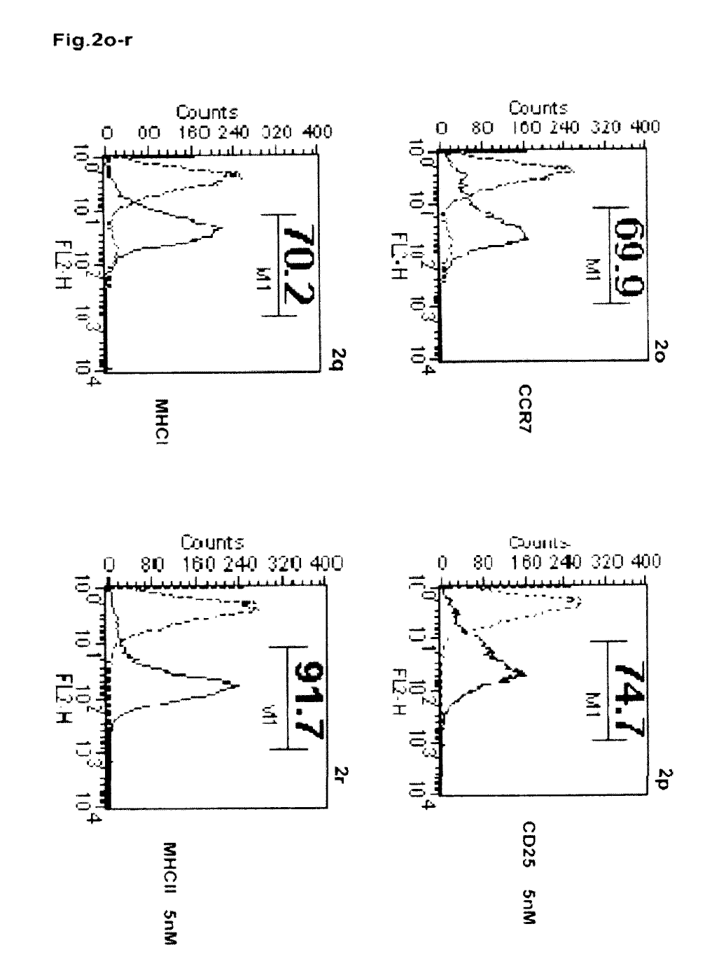

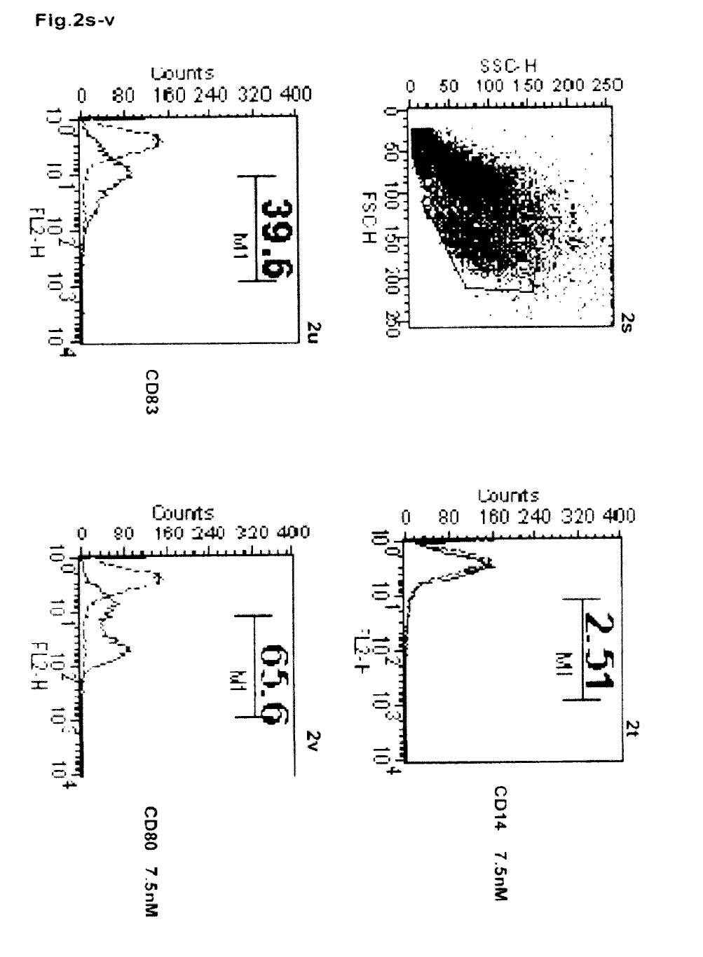

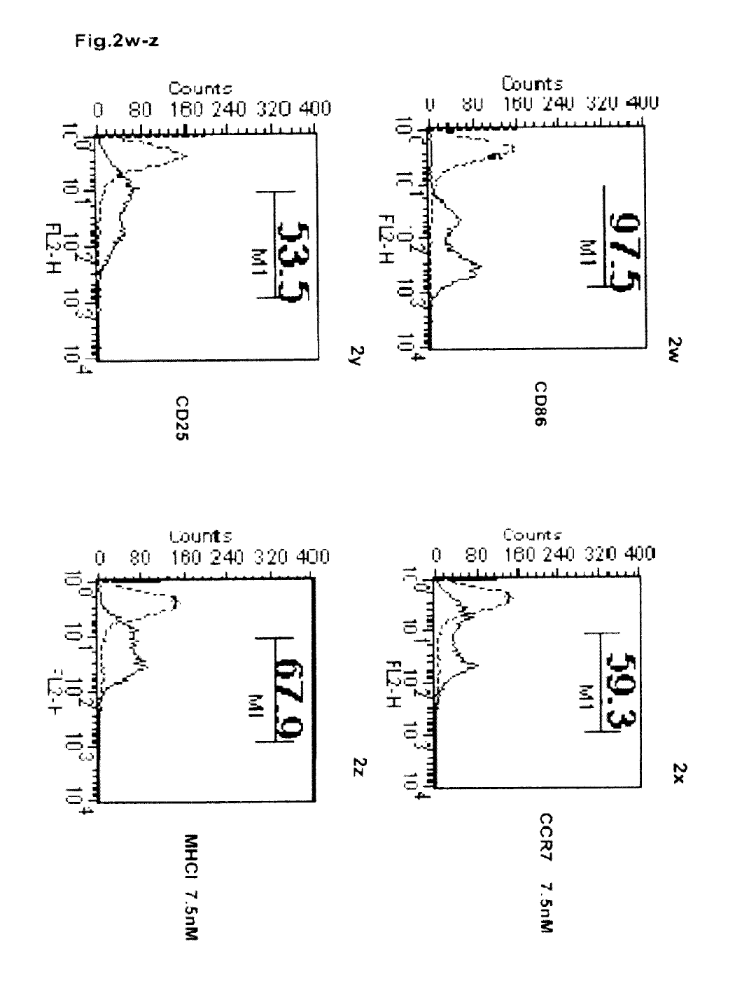

Interestingly, it was found, within the scope of the invention, that proteasome inhibitors block the maturation of DCs. It was possible to show this, in particular, in that the expression of typical surface molecules is inhibited (see FIGS. 1 and 2). Functionally, this means that the DCs are blocked in an immature stage and therefore cannot further induce any potent immune responses. On the contrary, the formation of tolerance mechanisms takes place. Therapeutically, this would be of great interest, particularly in cases of autoimmune diseases, asthma, allergies, as well as in avoiding tissue rejections or transplant rejections.

Proteasome inhibitors are natural or chemical substances that inhibit the activity of proteasome and are fundamentally known to a person skilled in the art. The first approved proteasome inhibitor, bortezomib, is effective against multiple myeloma, a malignant plasma cell illness. Proteasome inhibitors have been described for treating tumor illnesses (for example U.S. Pat. No. 6,083,903) and for treating viral infections (WO 02/30455).

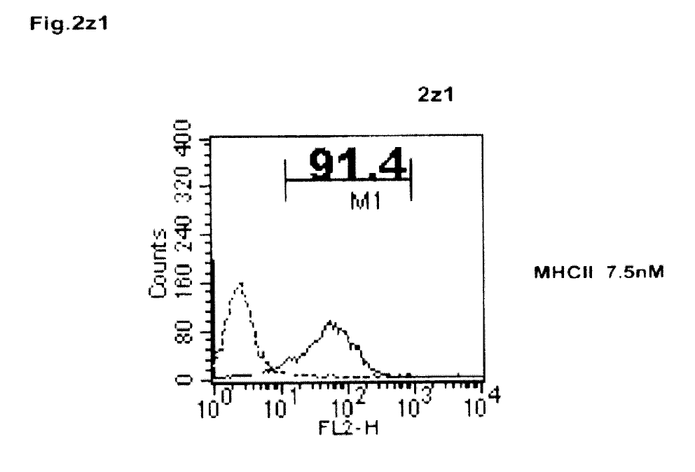

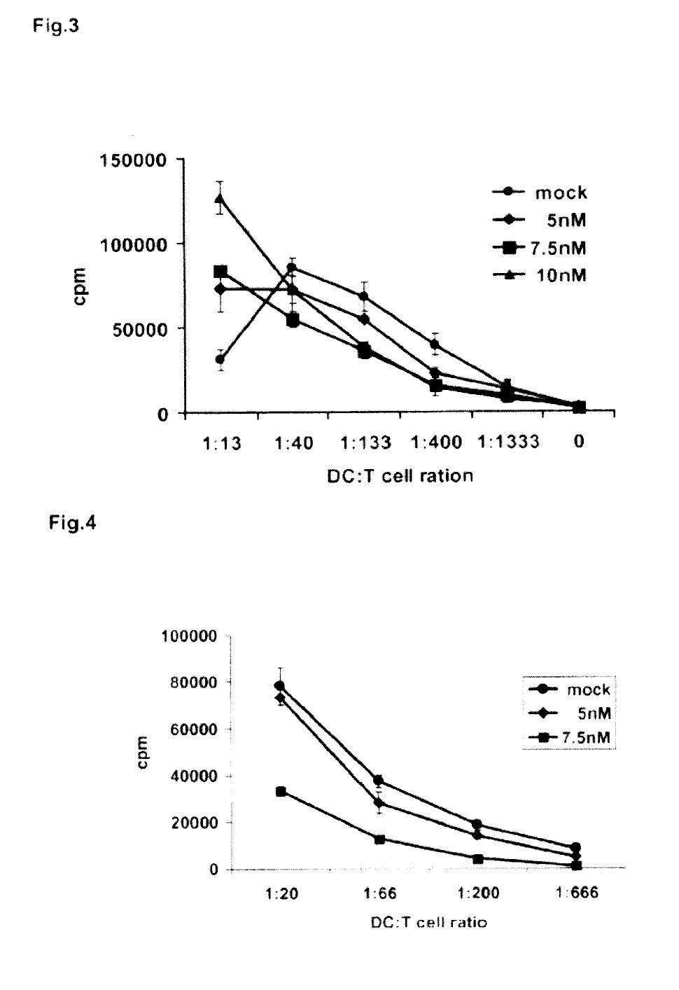

The T cell stimulatory capacity of mature DCs can be tested in vitro by means of the so-called “mixed leukocyte reaction” (MLR) assay. Interestingly, the proteasome inhibitors (XVL01 and Velcade®) are able to block DC-mediated T cell stimulation (see FIGS. 3 and 4). This functional test therefore reflects the phenotypic effects of the PI treatment—namely the blockade of full maturation of the DCs—very well.

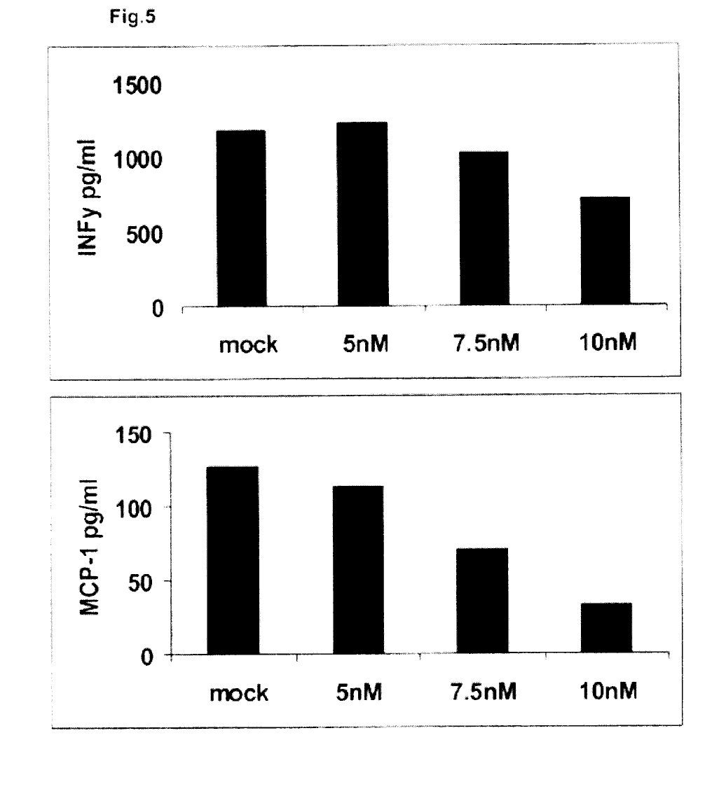

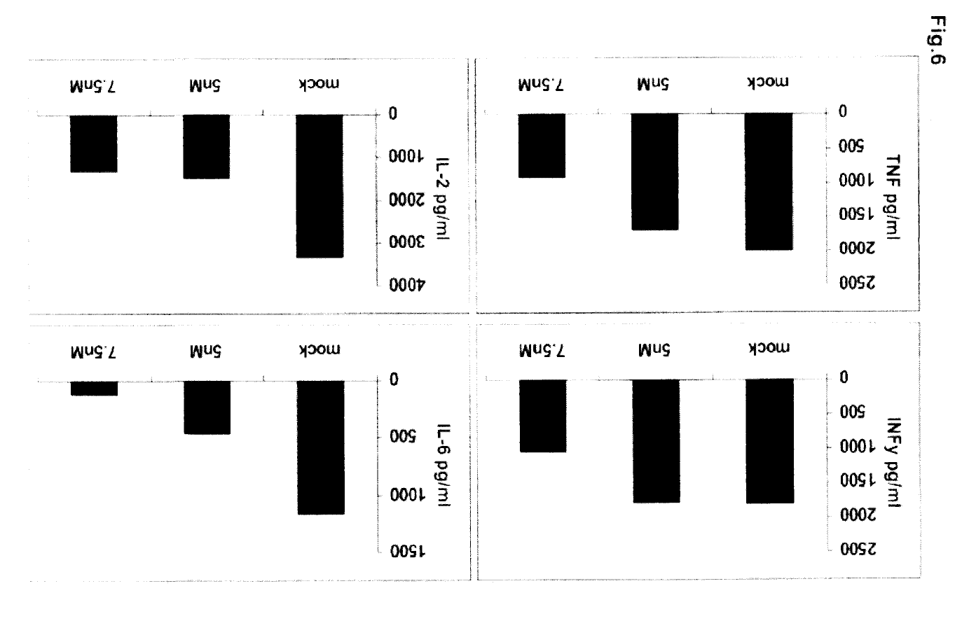

During DC-mediated T cell stimulation, the production of cytokines (among them IFN, IL-2, IL-6, TNF) takes place, which can be detected in the culture top fraction. Astonishingly, Velcade® was able to block this cytokine production, as a function of concentration (see FIGS. 5 and 6). This is a further indication/proof that PIs lead to a blockade of the immune-stimulating function of DCs.

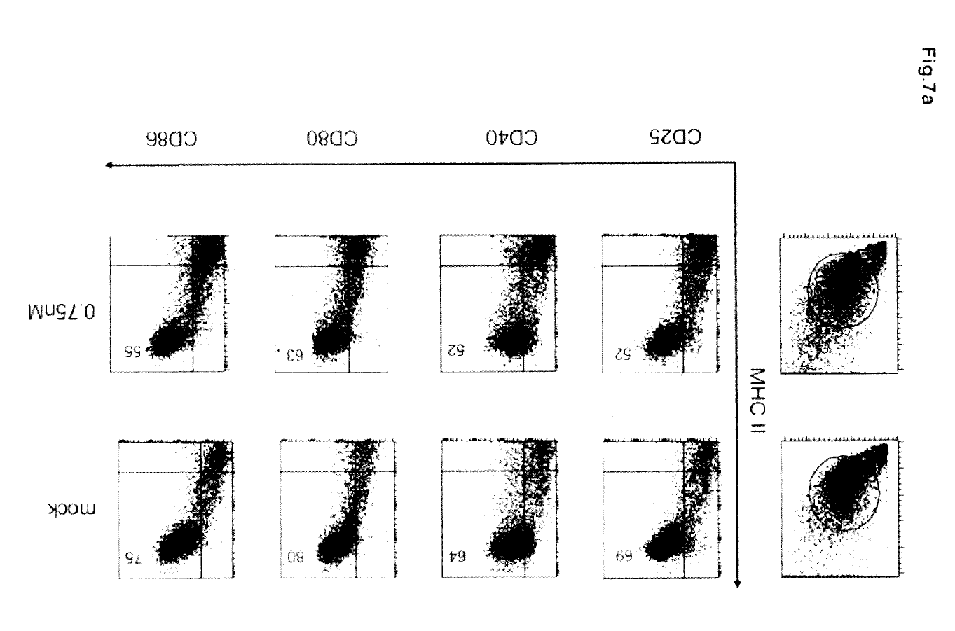

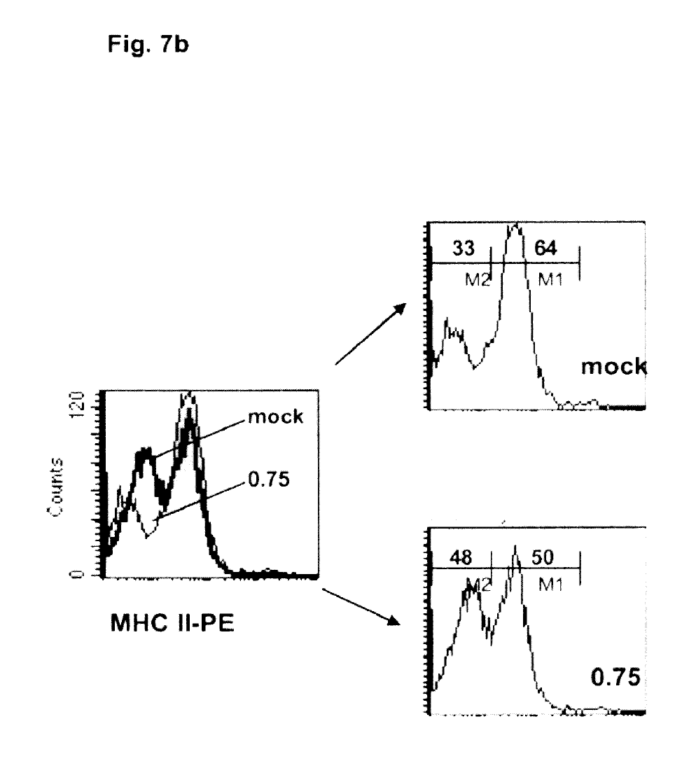

In order to test the effect of PIs also in vivo, Velcade® was applied to mice in vivo, then bone marrow was isolated and the typical bone marrow DCs were generated from this. As shown in FIG. 7, the application of Velcade® reduces the population of the mature DCs. This is because a downward shift occurs in the CD40, CD25, and CD83 highly expressing mature DC population, in other words toward a semi-mature or immature phenotype. This holds true not only for TNF-stimulated DCs but also for LPS-stimulated DCs.

The goal of this invention is therefore inhibiting DC maturation and thus T cell and B cell stimulation by means of PIs, and furthermore, as a result, the induction of tolerance mechanisms for treatment and/or prevention of illnesses (including autoimmunity, asthma, allergies, tissue rejection or transplant rejection), which are provoked by excessive immune reactions. PIs prevent complete DC maturation, which leads to a blockade of T cell proliferation and T cell activation. The invention therefore delivers the basis for treatment of illnesses that are characterized by excessive immune reactions. Tolerance mechanisms are induced by means of the treatment with PIs, among other things.

The object of the invention is the use of proteasome inhibitors for the production of agents for the treatment or prevention of allergies and/or asthma and/or tissue rejections or transplant rejections and/or autoimmune diseases. The concentration of the proteasome inhibitors lies in the nanomolar range, preferably in the range of 10 nM to 10 μM, with reference to the peripheral blood or the cytoplasm.

Substances that are isolated in natural form from microorganisms or other natural sources, proceed from natural substances by means of chemical modifications, or are produced totally synthetically, or are synthesized in vivo by means of gene therapy measures, or are produced in vitro or in microorganisms, can be used as proteasome inhibitors.

### a) Naturally Occurring Proteasome Inhibitors:

epoxomicin (epoxomycin) and eponemycin,
aclacinomycin A (also called aclarubicin),
lactacystin and its chemically modified variants, particularly the cell-membrane-penetrating variant “clasto-lactacystin β-lactone”,

### b) Synthetically Produced Proteasome Inhibitors:

modified peptide aldehydes, such as, for example, N-carbobenzoxy-L-leucinyl-L-leucinyl-L-leucinal (also called MG132 or zLLL), its boric acid derivative MG232; N-carbobenzoxy-Leu-Leu-Nva-H (called MG115); N-acetyl-L-leucinyl-L-leucinyl-L-norleucinal (called LLnL); N-carbobenzoxy-Ile-Glu(OBut)-Ala-Leu-H (also called PSI);
peptides that carry C-terminal α,β-epoxy ketones (also called epoxomicin/epoxomycin or eponemycin), vinyl sulfones (for example carbobenzoxy-L-leucinyl-L-leucinyl-L-leucin vinyl sulfone or 4-hydroxy-5-iodo-3-nitrophenylacetyl-L-leucinyl-L-leucinyl-L-leucin vinyl sulfone, also called NLVS), glyoxal or boric acid radicals (for example pyrazyl-CONH(CHPhe)CONH(CHisobutyl)B(OH)₂), also called “PS-431,” or benzoyl(Bz)-Phe-boroLeu, phenacetyl-Leu-Leu-boroLeu, Cbz-Phe-boroLeu); pinacol esters—for example benzyloxycarbonyl (Cbz)-Leu-Leu-boroLeu pinacol ester; and
peptides and peptide derivatives that carry C-terminal epoxy ketone structures are used as particularly suitable compounds; these include, for example, epoxomycin (molecular formula: C₂₈H₈₆N₄O₇) and eponemycin (molecular formula: C₂₀H₃₆N₂O₅);
chemically modified derivatives on the basis of naturally occurring, particularly a β-lactone derivative having the name PS-519 (1R-[1S, 4R, 5S]]-1-(1-hydroxy-2-methylpropyl)-4-propyl-6-oxa-2-azabicyclo[3.2.0]heptane-3,7-dione, molecular formula: C₁₂H₁₉NO₄), which is derived from the natural proteasome inhibitor lactacystin;
certain dipeptidyl boric acid derivatives, particularly compounds that are derived from the pyranozyl-phenyl-leucinyl boric acid derivative with the name “PS-341” (N-pyrazinecarbonyl-L-phenylalanine-L-leucin boric acid, molecular formula: C₁₉H₂₅N₄O₄).

These furthermore include the compounds (“PS-273” (morpholine-CONH—(CH-naphthyl)-CONH—(CH-isobutyl)-B(OH)₂) and its enantiomer PS-293, the compound PS-296 (8-quinolyl-sulfonyl-CONH—(CH-naphthyl)-CONH—(CH-isobutyl)-B(OH)₂); the compound PS-303 (NH₂(CH-naphthyl)-CONH—(CH-isobutyl)-B(OH)₂); the compound PS-321 (morpholine-CONH—(CH-naphthyl)-CONH—(CH-phenylalanine)-B(OH)₂); the compound PS-334 (CH₃—NH—(CH-naphthyl)-CONH—(CH-isobutyl)-B(OH)₂); the compound PS-325 (2-quinol-CONH—(CH-homo-phenylalanine)-CONH—(CH-isobutyl)-B(OH)₂); the compound PS-352 (phenylalaline-CH₂-CH₂-CONH—(CH-phenylalanine)-CONH—(CH-isobutyl)-B(OH)₂); the compound PS-383 (pyridyl-CONH—(CHpF-phenylalanine)-CONH—(CH-isobutyl)-B(OH)₂). All these compounds have already been described, among others in Adams et al. (1999).

Aside from epoxomicin and eponemycin, the proteasome inhibitors PS-519, PS-341, and PS-273 (developed by Millennium Pharmaceuticals, Inc., Cambridge, Mass. 02139, USA) have proven to be particularly suitable compounds. These proteasome inhibitors are very potent, very specific for proteasome, do not block any other cellular proteases, and have practically no side effects. The proteasome inhibitors PS-341 and PS-519 were furthermore tested both in animal models for pre-clinical trials and in humans (cancer patients) for clinical trials.

The autoimmune diseases are, for example, myasthenia gravis or multiple sclerosis, vasculitis, chronic inflammatory bowel diseases such as Crohn's disease or colitis ulcerosa, HLA B27-associated autoimmune pathologies such as Bechterew's disease or systemic lupus erythematosus (SLE), skin diseases such as psoriasis or pemphigus, or rheumatoid arthritis or diabetes mellitus.

A malfunction of the cellular immune system, in which dendritic cells and/or T cells and/or Th17 cells and/or B cells are involved, is treated or prevented by proteasome inhibitors. In particular, a malfunction of the cellular immune system in which T cells of the regulatory type are involved is treated or prevented by proteasome inhibitors.

The T cells of the regulatory type are naturally occurring regulator T cells (Treg) and/or IL-10 producing regulatory T cells of the Type 1.

The object of the invention is also the use of the proteasome inhibitors in combination with other immune-suppressive agents, such as, for example, rapamycin, CsA, mycophenolate mofetil (MMF), FK506, sCD83, and “immune-modulatory” antibodies, in optimal and/or suboptimal doses, in each instance, are used. From this, it is expected that toxic side effects of the individual substances will be reduced/avoided in the combination therapy.

The invention will be explained in greater detail in the following, using drawings, without being restricted to these examples.

The individual figures show:

FIG. 1 shows that the inhibition of the proteasome impairs the maturation of murine dendritic cells.

Murine DCs were incubated with different concentrations of the proteasome inhibitor Velcade® from Day 8 to Day 10, and were matured during the last 16 hours, in the presence of LPS. In order to determine the phenotypic DC maturation, in other words the increase in the expression level of specific cell surface molecules, the cells were subsequently characterized using monoclonal antibodies and FACS analysis. As shown in FIG. 1, Velcade® very clearly reduces the surface expression of CD25, CD40, CD80, CD86, and, in particular, of CD83, which are all typically upregulated during DC maturation. From this, it follows that the inhibition of the proteasome interferes with DC maturation. However, only completely matured DCs are able to induce potent immune responses. The control (mock) remained untreated.

FIG. 2 shows that the inhibition of the proteasome impairs the maturation of human dendritic cells.

Immature human DCs were mixed with two different concentrations of Velcade® on Day 5, and subsequently matured for another two days using the maturation cocktail. Subsequently, the expression of the surface molecules that are typical for the maturation of human DCs was analyzed. Velcade® led to a clear reduction in the expression of CD80, CCR7, CD25, MHC I, and especially of CD83. The control (mock) remained untreated. From this, it follows that the inhibition of the proteasome interferes with maturation in human DCs, as well.

It can be derived from FIG. 3 that the inhibition of the proteasome leads to a reduced DC-mediated T cell stimulation.

Allogeneic murine T cells were incubated with TNF-matured murine DCs for 72 hours, in the presence of three different concentrations of Velcade®. In contrast to control cells (mock), Velcade® reduces the DC-mediated T cell proliferation in a manner that is dependent on dose.

FIG. 4 describes that the inhibition of the proteasome in human DCs leads to a reduced DC-mediated T cell stimulation.

Human DCs were incubated with Velcade® during maturation and subsequently, the DC-mediated T cell stimulation was investigated by means of MLR assay. In contrast to the control cells (mock), Velcade®-treated DCs show a clear reduction in their stimulatory capacity.

From FIG. 5, it is evident that the inhibition of the proteasome leads to a reduced cytokine and chemokine production in murine DC/T cell co-cultures.

The production of INFy and MCP-1 was determined from cell culture top fractions of murine (DC:T) cell co-cultures. For this purpose, DCs were incubated with allogeneic T cells, in the presence of different concentrations of Velcade®, for 72 hours. The inhibition of the proteasome leads to a clearly reduced expression of INFy and MCP-1. The control (mock) remained untreated.

In FIG. 6, it is shown that the inhibition of the proteasome leads to a reduced cytokine production in human DC/T cell co-cultures.

Immature human T cells were incubated with Velcade®, matured for 48 hours, and subsequently cultivated together with allogeneic human T cells for another 72 hours. Subsequently, cell culture top fractions were taken and the release of pro-inflammatory cytokines was analyzed by means of CBA technology. The inhibition of the proteasome leads to a clearly reduced secretion of IL-2, IL-6, INFy, and TNF. The control (mock) remained untreated.

FIGS. 7a and 7b clearly show that the inhibition of the proteasome in vivo influences the maturation of murine DCs generated ex vivo.

C57B1/6 mice were injected with 0.75 mg/kg Velcade® i.v. three times. The injection took place within five days, with an interval of one day, in each instance. An hour after the last injection, removal of the bone marrow from femur and tibia took place to generate the murine DCs. On Day 8 of cultivation, the cells were matured with LPS. On the subsequent day, the expression of the surface molecules was investigated by means of FACS analysis. DCs from untreated animals (mock) showed the expression of the surface markers that is typical for mature DCs. In contrast to this, the analysis of the animals that were treated with Velcade® showed a reduction in the surface expression of CD25, CD40, CD80, and CD86 (FIG. 7a). Furthermore, the expression of MHC Class II molecules was also reduced (FIG. 7b). The percentage of MHC II highly expressing cells:MHC II interm. expressing cells was shifted in favor of the MCH II interm. expressing cells. These data show that the in vivo application of proteasome inhibitors impair DC maturation.

1.-8. (canceled)

9. A proteasome inhibitor which is capable of producing an agent for treatment or prevention of at least one disease selected from the group consisting of
a) an allergy,
b) asthma,
c) a tissue rejection or transplant rejection, and
d) an autoimmune disease.

10. Peripheral blood or cytoplasm, comprising:
the proteasome inhibitor according to claim 9 in a concentration of 1 nM to 100 μM, based on the peripheral blood or cytoplasm.

11. The proteasome inhibitor according to claim 9, wherein the autoimmune disease is at least one member selected from the group consisting of
a) myasthenia gravis,
b) multiple sclerosis,
c) vasculitis,
d) a chronic inflammatory bowel disease,
e) a HLA B27-associated autoimmune pathology,
f) systemic lupus erythematosus (SLE),
g) a skin disease,
h) pemphigus,
i) rheumatoid arthritis, and
j) diabetes mellitus.

12. The proteasome inhibitor according to claim 11, wherein the chronic inflammatory bowel disease is Crohn's disease.

13. The proteasome inhibitor according to claim 11, wherein the chronic inflammatory bowel disease is colitis ulcerosa.

14. The proteasome inhibitor according to claim 11, wherein the HLA B27-associated autoimmune pathology is Bechterew's disease.

15. The proteasome inhibitor according to claim 11, wherein the skin disease is psoriasis.

16. A method of treating or preventing a malfunction of a cellular immune system, comprising:
providing a proteasome inhibitor to treat or prevent said malfunction of said cellular immune system,
wherein dendritic cells and/or T cells and/or Th17 cells and/or B cells are involved in said malfunction of said cellular immune system.

17. The method according to claim 16, wherein regulatory T cells are involved in said malfunction of the cellular immune system.

18. The method according to claim 17, wherein regulatory T cells are naturally occurring regulatory T cells (Treg) and/or IL-10 producing regulatory T cells of Type 1.

19. The method according to claim 16, comprising providing said proteasome inhibitor in combination with another immune-suppressive agent.

20. The method according to claim 19, wherein the other immune-suppressive agent is at least one member selected from the group consisting of rapamycin, CsA, mycophenolate mofetil (MMF), FK506, sCD83, and “immune-modulatory” antibodies.

21. A mixture, comprising:
the proteasome inhibitor according to claim 9; and
another immune-suppressive agent.

22. The mixture according to claim 21, wherein the other immune-suppressive agent is at least one member selected from the group consisting of rapamycin, CsA, mycophenolate mofetil (MMF), FK506, sCD83, and “immune-modulatory” antibodies.

23. A method of treating or preventing a disease, comprising:
providing a proteasome inhibitor capable of producing an agent for treatment or prevention of at least one disease selected from the group consisting of
a) an allergy,
b) asthma,
c) a tissue rejection or transplant rejection, and
d) an autoimmune disease.

24. The method according to claim 23, wherein said proteasome inhibitor is in peripheral blood or cytoplasm and has a concentration of 1 nM to 100 μM, based on the peripheral blood or the cytoplasm.

25. The method according to claim 23, wherein the autoimmune disease is at least one member selected from the group consisting of
a) myasthenia gravis,
b) multiple sclerosis,
c) vasculitis,
d) a chronic inflammatory bowel disease,
e) a HLA B27-associated autoimmune pathology,
f) systemic lupus erythematosus (SLE),
g) a skin disease,
h) pemphigus,
i) rheumatoid arthritis, and
j) diabetes mellitus.

26. The method according to claim 25, wherein the chronic inflammatory bowel disease is Crohn's disease.

27. The method according to claim 25, wherein the chronic inflammatory bowel disease is colitis ulcerosa.

28. The method according to claim 25, wherein the HLA B27-associated autoimmune pathology is Bechterew's disease.

29. The method according to claim 25, wherein the skin disease is psoriasis.

30. The method according to claim 23, comprising providing said proteasome inhibitor in combination with another immune-suppressive agent.

31. The method according to claim 30, wherein the other immune-suppressive agent is at least one member selected from the group consisting of rapamycin, CsA, mycophenolate mofetil (MMF), FK506, sCD83, and “immune-modulatory” antibodies.
